(19) United States
(12) Patent Application Publication
(10) Pub. No.: US 2026/0134968 A1
(43) Pub. Date: May 14, 2026

(54) SYSTEMS AND METHODS OF GENERATING DYNAMIC ACTIVITY RECOMMENDATIONS BASED ON MACHINE LEARNING ARCHITECTURE

(71) Applicant: Train Fitness Inc., Toronto (CA)

(72) Inventors: Robert Andrew JUST, Toronto (CA); Vivek VERMA, Delhi (IN)

(21) Appl. No.: 19/389,966

(22) Filed: Nov. 14, 2025

Related U.S. Application Data
(60) Provisional application No. 63/720,354, filed on Nov. 14, 2024.

(51) Int. Cl. G16H 20/30; G16H 40/60

(52) U.S. Cl. CPC G16H 20/30; G16H 40/60

(57) ABSTRACT

A fitness tracking system can include at least one processor and non-transitory memory storing instructions which, when executed by the at least one processor, cause the at least one processor to perform operations including receiving, from one or more wearable devices having sensors for detecting motion of a user, first data characterizing raw motion of a user during an exercise activity, determining, using a first artificial intelligence (AI) model, based on the first data, a readiness metric associated with the exercise activity, determining, using a second AI model, based on the readiness metric, an exercise plan for the user, and providing second data characterizing the exercise plan.

### CROSS-REFERENCE TO RELATED APPLICATIONS

This application claims the benefit of U.S. Provisional Patent Application No. 63/720,354, filed on November 14, 2024, entitled “Fitness Tracking System and Method of Operating the Same,” the entirety of which is hereby incorporated by reference.

### FIELD

Embodiments of the present disclosure generally relate to fitness tracking systems, and in particular to systems and methods of generating activity recommendations.

### BACKGROUND

Fitness tracking systems are designed to monitor, record, and analyze physical activity to support users in achieving their health and fitness goals. These systems provide insights into exercise habits, help track progress over time, and encourage consistent engagement with fitness routines.

Existing fitness tracking systems typically include a mobile computing device, such as a smart phone, which serves as a central hub for collecting and managing exercise data. Users often manually input workout-related information into the mobile device, including the types of exercises performed, the number of repetitions, and the amount of weight used. In addition to manual data entry, some fitness tracking systems incorporate wearable computing devices, such as smart watches or fitness tracking bands. These wearable devices collect motion data while the user is exercising and enable the tracking of physiological parameters, such as heart rate, in real time during physical activity.

### SUMMARY

The present disclosure describes fitness tracking systems and methods of operating the same. In some embodiments, fitness tracking systems and methods may be configured to provide exercise predictions or exercise activity recommendations to exercise users.

Exercise users may have a plurality of varying attributes, including physical size, physiological capacity or capability, health history, or biomarker history, among other attributes. A generated base line exercise activity plan may not be a one-size-fits-all exercise activity plan that is optimal for respective exercise users. It may be desirable to provide systems and methods for generating user-specific exercise activity recommendations based on user attributes or biomarker data sets associated with respective users on a substantial real-time basis. Such systems and methods may be based on machine learning architecture.

In some aspects, fitness tracking systems are described. In some embodiments, a system can include at least one processor and non-transitory memory storing instructions which, when executed by the at least one processor, cause the at least one processor to perform operations including receiving, from one or more wearable devices having one or more sensors for detecting motion of a user, first data characterizing raw motion of a user during an exercise activity, determining, using a first artificial intelligence (AI) model, based on the first data, a readiness metric associated with the exercise activity, retrieving an exercise plan for the user based on the readiness metric generated based on a second AI model, and providing second data characterizing the exercise plan.

In some embodiments, the one or more wearable devices include a smart watch, a fitness tracking band, wireless headphones, or wireless earphones.

In some embodiments, the at least one processor includes a first processor associated with a first device and a second processor associated with a second device. The first AI model can be implemented by the first processor and the second AI model can be implemented by the second processor. The first processor and the second processor can be communicatively coupled over a wireless network.

In some embodiments, the first AI model includes one or more convolutional neural network (CNN) layers, one or more long short-term memory (LSTM) layers, one or more embedding layers, or a combination thereof. In some embodiments, the second AI model includes at least one large language model (LLM) layer.

In some embodiments, the operations further include determining, by the first AI model, one or more physiological characteristics of the user. The readiness metric can be determined based on the one or more physiological characteristics.

In some embodiments, determining the readiness metric includes determining an effort score representing an amount of effort exerted by the user during the exercise activity.

In some embodiments, determining the readiness metric includes determining one or more motion metrics characterizing motion of the user or a body part of the user during the exercise activity. The one or more motion metrics can include metrics indicating an acceleration of the user or the body part, a velocity of the user or the body part, a time under tension of the user or the body part, a power exerted by the user or the body part, a motion path traversed by the user or the body part, a stability of the user or the body part, or a combination thereof.

In other aspects, corresponding systems and devices, and logic structures such as machine-executable coded instruction sets for implementing the described systems, devices, and methods are provided.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrates a fitness tracking platform, in accordance with embodiments of the present disclosure;

FIG. 2 illustrates a block diagram of a fitness tracking platform, in accordance with embodiments of the present disclosure;

FIG. 3 illustrates an example smart watch device worn by a user partaking in weightlifting exercises, in accordance with embodiments of the present disclosure;

FIG. 4 illustrates a mobile computing device carried by a user in a garment pocket in varying orientations during an exercise activity, in accordance with embodiments of the present disclosure;

FIG. 5 illustrates a block diagram of a wearable computing device, in accordance with embodiments of the present disclosure;

FIG. 6 illustrates a flowchart of a method of generating exercise activity recommendations, in accordance with embodiments of the present disclosure;

FIG. 7 illustrates a diagram of an artificial intelligence model architecture, in accordance with embodiments of the present disclosure; and

FIG. 8 illustrates a flowchart of a method of generating fitness exercise recommendations based on machine learning architecture, in accordance with embodiments of the present disclosure.

### DETAILED DESCRIPTION

Users are frequently drawn to fitness tracking systems because they seek tools for facilitating their progression toward their personal fitness goals. Effective progression generally requires developing and following a tailored exercise plan. However, most conventional systems require users to manually log their workouts and, as a result, are capable of receiving only basic details such as exercise type, duration, and repetitions. Such manually entered information is often insufficient for developing meaningful insight into how well the user executed a given movement or the level of effort exerted during the activity. While some platforms may allow users to input subjective assessments of their form quality or perceived exertion, self-reported data is often biased, inconsistent, and prone to inaccuracies. As a result of this inability to collect robust exercise-related data, existing fitness tracking systems are generally not equipped to generate personalized exercise plans that reflect the user’s actual performance and physical condition. This limitation significantly hampers the effectiveness of conventional systems in guiding users toward improved fitness outcomes.

Described herein are embodiments of fitness tracking systems that utilize one or a combination of artificial intelligence (AI) models to monitor and evaluate exercise activities performed by a user and to generate customized exercise recommendations based on the evaluation. The combination of AI models can include a specialized exercise activity assessment model configured to detect and assess exercise movements in real time using motion data captured by wearable devices associated with the user, such as smartwatches, fitness bands, or wireless headphones. This evaluation can include an assessment of movement quality, such as an assessment of the user’s form while performing an exercise, as well as an assessment of user effort, such as a gauge of how strenuous the exercise was for the user. Output from the exercise activity assessment model can then be provided to a second AI model, such as a large language model (LLM), which can process the output to generate tailored exercise recommendations for the user.

Embodiments of the described systems can provide recommendations both during an ongoing workout session and for future workout sessions. For example, during a workout, if the system detects that the user is expending unusually high levels of effort on a particular exercise, the system may recommend that the user reduce the weight used or perform fewer repetitions during a subsequent exercise set. For future sessions, the system can analyze historical workout data in conjunction with contextual information such as the user’s physiological characteristics, current physical state, and fitness goals to recommend specific exercises. These recommendations can include guidance on metrics such as the number of repetitions, sets, or resistance levels for each suggested activity, enabling the user to follow a plan optimized for their performance and progress.

Embodiments of the AI models employed by the described systems enable motion data collected from wearable computing devices to be processed in real time. This motion data is often complex and highly variable, reflecting (often subtle) differences in movement patterns across individuals with diverse physiological characteristics such as height, weight, and gender. Accurately identifying specific exercises, counting repetitions, assessing movement quality, and evaluating user effort requires computational analysis that accounts for these variations. The AI models can be implemented with a large number (e.g., millions) of parameters—including nodes, weights, and biases—that enable the model to execute intensive, multi-dimensional computations with high efficiency. As a result, the described systems can rapidly and accurately analyze large and heterogeneous datasets in view of a unique combination of physiological variables, producing exercise- specific insights that cannot be reliably or feasibly generated in real time through human observation or mental calculation alone.

Certain implementations will now be described to provide an overall understanding of the principles of the structure, function, manufacture, and use of the devices and methods disclosed herein. One or more examples of these implementations are illustrated in the accompanying drawings. Those skilled in the art will understand that the devices and methods specifically described herein and illustrated in the accompanying drawings are non-limiting implementations and that the scope of the present invention is defined solely by the claims. The features illustrated or described in connection with one implementation may be combined with the features of other implementations. Such modifications and variations are intended to be included within the scope of the present invention.

Mobile computing devices and wearable computing devices may be carried or worn by users during one or more activities. For example, smartphones may be commonly carried by a user in a garment pocket. Smart watches may be worn by a user throughout the course of a day and, in some situations, while sleeping. Wireless audio devices such as ear buds may be worn while exercising, among other activities.

In some embodiments, such mobile computing devices and wearable computing devices may include one or more sensors configured to monitor motion-related or environment-related data associated with a computing device. In some embodiments, sensors may include accelerometers, gyroscopes, pedometers, magnetometers, or barometers, among other examples.

Embodiments of fitness tracking systems described herein may be configured to obtain sensor data sets for determining motion or environment conditions associated with a computing device. For example, motion may include movement such as tilt, shake, rotation, acceleration, or swing. In some situations, determined motion or environmental conditions may correspond to user input, user movement, or the physical environmental conditions associated with the user of the computing device. In some embodiments, environment conditions may be associated with pre-activity or post-activity movements, 3^{rd} party data sets associated with geolocation data, magnetometer data associated with detecting equipment devices, among other examples to be described in the present disclosure.

Based on one or more of determined user movement or physical environmental conditions, the computing device may be configured to predict or infer a type of activity being undertaken by a user.

Exercise users may be associated with a plurality of varying attributes, including physical size, physiological capacity or capability, health history, or biomarker history, among other attributes. In some scenarios, it may be challenging to generate a single baseline exercise activity plan for a spectrum of exercise users. That is, a baseline exercise activity plan may not be a one-size-fits-all exercise activity plan that is optimal for respective users. It may be desirable to provide user-specific exercise activity recommendations based on user attributes or biomarker data sets associated with respective users.

Some embodiments disclosed herein may be based on a user donning a sole or preferred fitness tracking device, such as a smart watch or other computing device band on the user’s limb during exercise activity. The sole or preferred fitness tracking device may be configured to generate exercise activity recommendations, exercise tracking predictions, determine exercise repetition counts, among other examples of operations.

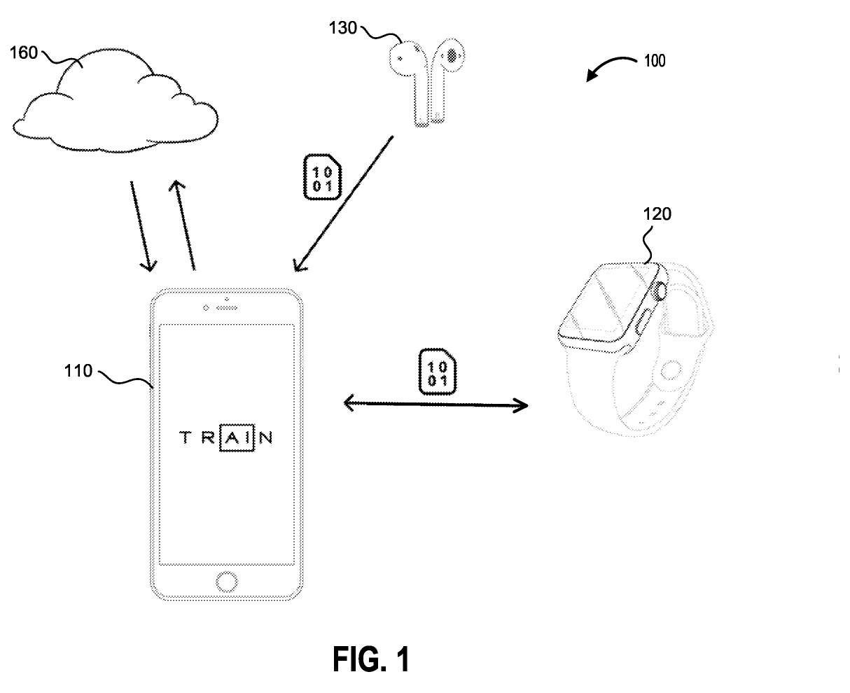

Reference is made to FIG. 1, which illustrates a fitness tracking platform 100, in accordance with an embodiment of the present disclosure. The fitness tracking platform 100 may include a mobile computing device 110. In some embodiments, the mobile computing device 110 may be a smartphone or a pocket personal computer, among other examples, and the mobile computing device 110 may be configured to transmit or receive, via a network, data messages to / from one or more client devices.

In the illustrated embodiment of FIG. 1, the mobile computing device 110 may be configured to conduct operations of machine learning models for generating exercise predictions or determining exercise repetition counts, among other operations, based on sensor data generated at the plurality of other devices of the fitness tracking platform 100. It may be contemplated that operations of machine learning models may be distributed, solely or in part, to other devices of the fitness tracking platform 100.

In some embodiments, client devices may include a smartwatch device 120, an audio device 130, or other wearable computing devices, such as fitness tracking bands, smart eyewear, among other examples. In FIG. 1, two example client devices may be the smartwatch device 120 and a pair of earbud-type audio devices 130. In some other embodiments, the fitness tracking platform 100 may include a single client device or may include any other number of client devices.

In some embodiments, the fitness tracking platform 100 may be configured to transmit or receive, via the network, data messages to and from a data server 160. In some embodiments, the data server 160 may be a centralized application server, Software as a Service (SaaS) computing platform, among other examples.

As will be described with reference to examples in the present disclosure, the data server 160 may be configured with operations to manage features of the fitness tracking platform 100, to provide social media-based functionality for a plurality of users, or to provide distributed computing operations for machine learning models for predicting or inferring types of activity based on data sets representing user movement or physical environmental conditions corresponding to the user. The data server 160 may be configured with other operations.

Embodiments of the fitness tracking system 100 may include machine learning models for generating predictions of type of user activity and for determining exercise activity statistics to provide feedback to the user. The machine learning models may be trained by training data sets prepared based on sensor data sets associated with video footage of users partaking in exercise activities.

For example, training data sets may be generated by obtaining sensor data from a smart watch, and simultaneously recording and associating video footage of a user conducting exercises (e.g., running, bench presses, pushups, rowing machine exercises, etc.). To illustrate, the sensor data may represent physiological user motion based on gyroscope sensor data and/or accelerometer sensor data recorded at a rate of up to 100 samples per second. Other sensor data sampling rates may be used.

In some embodiments, operations of the machine learning models for generating predictions and for generating exercise activity statistics may be conducted at the mobile computing device 110, at the data server 160, or a combination of devices.

In some embodiments, the training data sets may be augmented or altered for performing feature engineering and to train the machine learning models. For example, subsets of obtained sensor data may be altered to simulate potential exercise behaviors of fitness enthusiasts. Feature engineering operations may include increasing the speed at the front end of an exercise activity set or decreasing the speed at the back end of an exercise activity set to simulate explosive activity repetitions and fatigue, respectively. In some other examples, feature engineering operations may include operations to rotate or transform sensor data signals to simulate different user body types, body builds, among other user characteristics.

In some embodiments, the machine learning models may be configured to detect exercise activity types when the exercise activity begins or when the exercise activity ceases. In some embodiments, the machine learning models may be configured to track the number of exercise activity repetitions.

In some other embodiments, the machine learning models may be configured to recognize or generate additional exercise activity types. The recognition or generation of additional exercise activity types may include detecting a user perform the “new” exercise activity for at least 5 sets of repetitions. For example, a user may begin a new sequence of exercise motions (e.g., “twisty-jump-spin-lunge”) and may want to track this sequence of physical activity. The machine learning models may generate custom motion filters for dynamically detecting and tracking such “new” exercise activity.

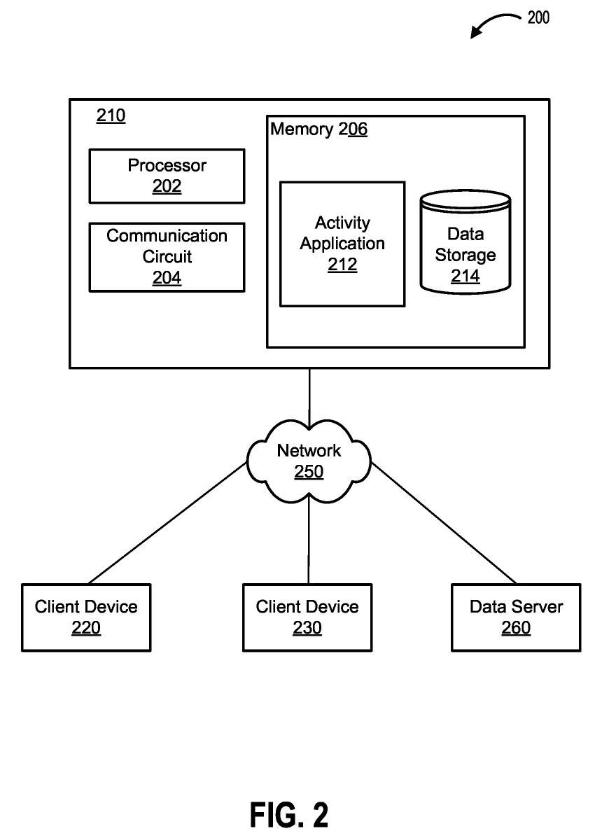

Reference is made to FIG. 2, which illustrates a block diagram of a fitness tracking platform 200, in accordance with embodiments of the present disclosure. The block diagram of the fitness tracking platform 200 may be an example of the fitness tracking platform 100 illustrated in FIG. 1.

A mobile computing device 210 may be configured to transmit or receive, via a network 250, data messages to or data messages from client devices (220, 230) or a data server 260. Two example client devices (220, 230) and a sole data server 260 are illustrated in FIG. 1. In some other examples, any number of client devices or subscription devices may be used.

To illustrate features of the fitness tracking system 200, the mobile computing device 210 may be a smart phone device. The smart phone device may be configured to communicate with client devices (220, 230) such as a smart watch device worn by a user or a pair of ear bud devices via the network 250. The smart phone device may be configured to communicate with the data server 260, such as a SaaS server or similar computing device, via the network 250.

In some embodiments, the mobile computing device 210 may communicate with the respective client devices (220, 230) or the data server 260 based on a common network communication protocol or based on different network communication protocols. For example, communication between the mobile computing device 210 and the client devices (220, 230) may be based on near-field communication protocols and the communication between the mobile computing device 210 and the data server 260 may be based on other wired or wireless network mediums.

The network 250 may include a wired or wireless wide area network (WAN), local area network (LAN), a combination thereof, or other networks for carrying telecommunication signals. In some embodiments, network communications may be based on HTTP post requests or TCP connections. Other network communication operations or protocols may be used.

In some embodiments, the network 250 may include near-field communication networks, such as Bluetooth™ networks, among other examples. In some examples, the network 250 may include the Internet, Ethernet, plain old telephone service line, public switch telephone network, integrated services digital network, digital subscriber line, coaxial cable, fiber optics, satellite, mobile, wireless, SS7 signaling network, fixed line, local area network, wide area network, or other networks, including one or more combination of the networks.

The mobile computing device 210 includes a processor 202 to implement processor-readable instructions that, when executed, configure the processor 202 to conduct operations described herein. The mobile computing device 210 may be configured to obtain data sets representing sensor data associated with physiological motion of a user and to dynamically generate predictions of user activity type or activity metrics in substantial real-time to the user. Other example operations will be described herein.

The processor 202 may be a microprocessor or a microcontroller, a digital signal processing processor, an integrated circuit, a field programmable gate array, a reconfigurable processor, or combinations thereof.

The mobile computing device 210 includes a communication circuit 204 configured to transmit or receive data messages to or from other computing devices, to access or connect to network resources, or to perform other computing applications by connecting to a network (or multiple networks) capable of carrying data. In some examples, the communication circuit 204 may include one or more busses, interconnects, wires, circuits, or other types of communication circuits. The communication circuit 204 may provide an interface for communicating data between components of a single device or circuit.

The mobile computing device 210 includes memory 206. The memory 206 may include one or a combination of computer memory, such as random-access memory, read-only memory, electro-optical memory, magneto-optical memory, erasable programmable read-only memory, and electrically-erasable programmable read-only memory, ferroelectric random-access memory, or the like. In some embodiments, the memory 206 may be storage media, such as hard disk drives, solid state drives, optical drives, or other types of memory.

The memory 206 may store an activity application 212 including processor-readable instructions for conducting operations described herein. In some examples, the activity application 212 may include operations for conducting machine learning operations associated with activity type prediction, operations associated with a recommendation application for providing exercise training recommendations in substantial real-time to a user during user exercise activity, or other example operations described in the present disclosure.

The mobile computing device 210 includes data storage 214. In some embodiments, the data storage 214 may be a secure data store. In some embodiments, the data storage 214 may store data sets received from the client devices (220, 230) or the data server 260. The data store 214 may be configured as a repository for data sets representing sensory data or other associated metadata from data-rich devices, such as smart watch devices, ear bud devices, smart garments, fitness tracker bands, among other devices (e.g., client devices 220, 230 or the data server 260). In some embodiments, the data store 214 may be include a features store. A features store may be a local cache of user motion data representing motion data of one or more custom exercises. The features store may include features which may be a raw data representation of exercise motion.

The client devices 220, 230 may be wearable computing devices such as smart watches, fitness tracking bands, smart eyewear, smart garments, wireless audio devices, among other examples. The wearable computing devices may be devices that a user may have adopted to wear routinely for one or more user exercise activities, such as while working out at a gym or exercising outdoors. The respective client devices 220, 230 may be configured as data-rich devices including sensors for detecting motion, patterns inherent in a sequence of motions, identifiable characteristics of detected motion, physical environment conditions, among other sensor-acquired data.

The respective client devices 220, 230 may include a processor, a memory, or a communication interface, similar to the example processor, memory, or communication interface of the mobile computing device 210. In some embodiments, the respective client devices 220, 230 may be computing devices associated with a local area network for transmitting or receiving signals to or from the mobile computing device 210. The local area network may include a wireless local area network or near-field communication networks such as Bluetooth™ or the like.

The data server 260 may be a computing device such as a data server, database device, or other data storing system for providing remote computing resources. For example, the data server 260 may conduct operations for managing or combining data sets from a plurality of mobile computing devices 210, where respective mobile computing devices 210 may conduct operations of the activity application 212.

In some embodiments, the data server 260 may be configured to provide gamification features or social media-related features to a plurality of users. For example, users of respective smartphone devices may opt to “follow” other users within a social network and compare exercise activity metrics with other users. In some examples, providing social-media related features can foster a community associated with exercise and healthy user lifestyles. In some embodiments, shared exercise activity metrics may be shared or kept private from other respective users.

In some embodiments, the data server 260 may provide gamification features to generate community competitions to incite friendly rivalry, and exercise activity level achievement rewards may be provided when users reach specific exercise activity level goals. In some embodiments, social media-related features may provide “leader boards” based on social groups associated with fitness centers attended, user profession, geographical location, age, or custom user groups. Social media-related features may motivate users to strive for and achieve fitness goals generated by the activity application 212 or created by respective users.

Example operations of the data server 260 described above may, in some embodiments, be conducted on the mobile computing device 210, or may be conducted on a combination of the data server 260 and the mobile computing device 210.

Embodiments of fitness tracking systems described herein may be configured to generate or obtain data sets representing sensor data from one or more data-rich devices (e.g., smartphone or wearable computing devices), dynamically track user exercise activity while the user may be at a gym, generate based on machine learning models predictions of specific user exercise activity type, and/or dynamically generate recommendations to the user during the user exercise activity. As such, embodiments of fitness tracking systems described herein may provide features of a virtual strength-training application for automatically identifying whether a user is doing squats or bench presses, push-ups or sit ups, or tally exercise repetitions. Further, the fitness tracking systems may be configured to generate user exercise activity metrics, such as rest time, range of motion, velocity, or the like, that may be transmitted to a live coach or trainer for progress monitoring.

To illustrate embodiments, the following examples illustrate a user who may be wearing or carrying at least one of a smart watch (e.g., Apple Watch™, or the like), wireless ear buds having one or more motion sensors therein (e.g., Apple AirPods™, or the like), or a smart phone (e.g., Apple iPhone™, Android-based smart phone, or the like) during an exercise or workout session. During a user’s exercise activity, the smart phone may conduct operations of an activity application 212 (FIG. 2) for obtaining substantially continuous, real-time data sets from the smart watch, wireless ear buds, or other user wearable devices for generating in substantial real-time predictions of the type of exercise activity that the user may be partaking in. The activity application 212 may provide one or more of the above-described generated predictions as feedback to the user via graphical user interfaces or audio interfaces.

In some embodiments, the activity application 212 may conduct operations to automatically detect the start of a workout activity and an end of the workout activity, without obtaining user input to indicate the start or conclusion of the workout activity. Upon detecting a start of a workout activity, the activity application 212 may be configured to dynamically generate a user interface for display at the smart watch or the smart phone. The user interface may be configured to provide a list of at least one predicted exercise associated with the machine learning model output, and the user may provide feedback on whether the predicted exercise activity predictions may be accurate. Such user feedback may be utilized for improving or training the machine learning model.

The activity application 212 may in substantial real-time determine one or a plurality of exercise activity statistics or details, such as range of user motion, velocity, acceleration, detected user rest time, physiological metrics of the user (e.g., heart rate, etc.) for providing the user with guidance or motivation through the exercise activity. Upon detecting a conclusion of the activity exercise or a repetition set, the activity application 212 may generate a summary of the user’s activity exercise. Data sets generated during user exercise activity may form the basis of training data sets for improving machine learning model output and may form the basis for providing future exercise activity guidance.

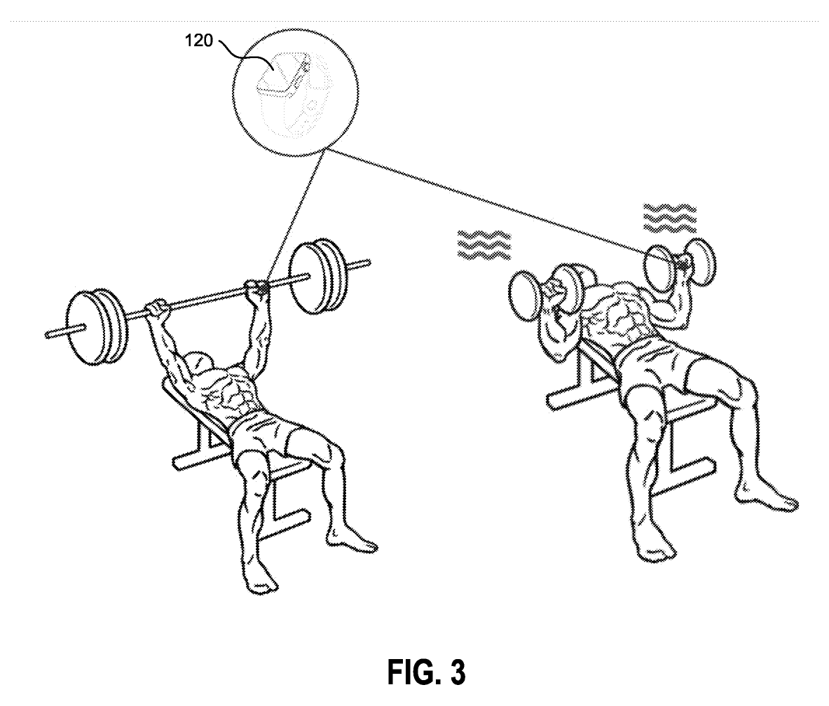

Reference is made to FIG. 3, which illustrates an example of a smart watch device 120 (FIG. 1) worn by a user partaking in weightlifting exercises, in accordance with embodiments of the present disclosure. The user may be wearing the smart watch device 120 on a wrist of the user.

In some embodiments, the smart watch device 120 may include one or more sensors configured to detect motion representing user movement or physical environment conditions. For example, the smart watch device 120 may include one or more of an accelerometer, a gyroscope, a magnetometer, or other sensors for detecting acceleration, gyroscopic motion, gravity, or magnetic field during exercise activity. Data sets associated with the detected motion may be for deriving or predicting the exercise activity type by the user.

FIG. 3 illustrates the user doing weightlifting exercises, such as bench presses with a barbell and, alternatively, with dumbbells. As the user may be wearing a smart watch device 120, the smart watch device 120 may generate a series of sensor data, and the series of sensor data may be used for generating predictions on the type of weightlifting exercise by the user.

Although both drawings in FIG. 3 show a user conducting bench press exercises, the respective drawings illustrate the user conducting bench press exercises based on different equipment. In some embodiments, the activity application 212 (FIG. 2) may conduct operations for distinguishing the type of activity / equipment used by the user based on characteristics derived from sequences of sensor data.

In one example, the user may be conducting bench press exercises with a barbell. In another example, the user may be conducting bench press exercises with dumbbells. The user’s wrist motion when conducting bench presses with a barbell may be different than the user’s wrist motion when conducting bench presses with dumbbells, at least because there may be greater variation in wrist movement when pushing up on dumbbells as compared to wrist movement when pushing up on a barbell.

In some situations, a user may be conducting one or more exercises associated with common physiological motion characteristics but may be different in user positioning. For example, a user partaking in bench press exercises with a barbell may exhibit upper body or arm motion, as detected by one or more sensors by a smart watch, similar to upper body or arm motion exhibited with the user partaking in overhead press exercises. However, the user partaking in bench press exercises may be lying down on a bench, whereas the user partaking in overhead press exercises may be in a partially upright, standing position. It may be beneficial to provide fitness tracking system features to combine data sets from two or more client devices to predict or infer an activity type with increased confidence levels / scores, thereby being able to increase exercise prediction accuracy to distinguish exercise activities having common physiological motion characteristics, but that may nonetheless be different exercise activities.

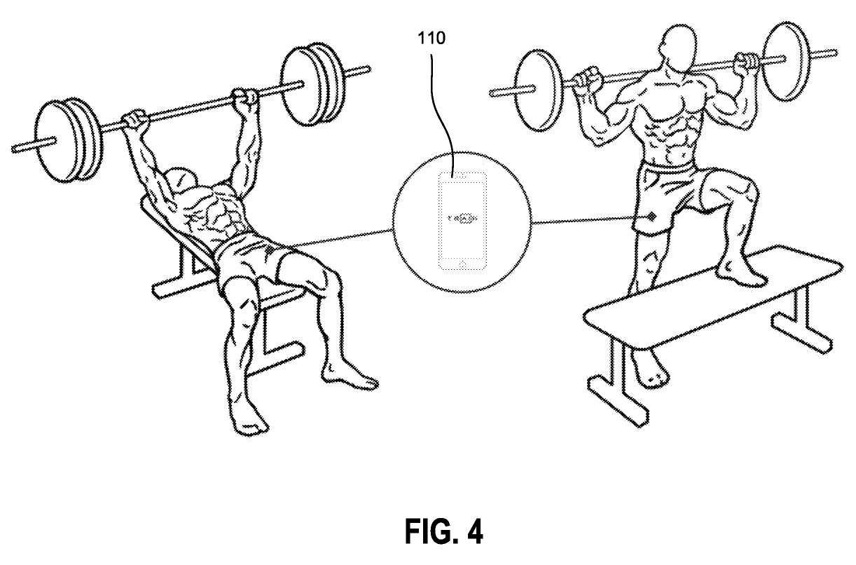

Reference is made to FIG. 4, which illustrates the mobile computing device 110 (FIG. 1) carried by the user in a garment pocket, in accordance with embodiments of the present disclosure. In FIG. 4, the user may also be wearing a smart watch device (not explicitly illustrated in FIG. 4).

The mobile computing device 110 may be in communication with the smart watch device, and may obtain substantially continuous, real-time data sets from the smart watch device representing physiological motions of the user’s wrist / arm movement.

The drawings in FIG. 4 illustrate the user partaking in bench press exercises and the user, subsequently, partaking in standing press exercises. The mobile computing device 110 may conduct operations of the fitness application 112 (FIG. 1) for predicting that the user is partaking in one of either bench press exercises or standing press exercises. In the present example, the motion detected by the smart watch device when the user partakes in bench press exercises or the standing press exercises may be similar. The mobile computing device 110 may generate a prediction on the type of exercise being conducted and may display the predictions on a user interface for the user to confirmation input on.

To increase confidence levels / scores associated with predicting the exercise activity by the user, the computing device 110 may in some embodiments generate predictions based on data sets from two or more computing devices. In the example illustrated in FIG. 4, the orientation of the mobile computing device 110 in three dimensional space may be different when: (i) the user is lying on a bench when partaking bench press exercises; and (ii) the user is in a substantially standing position when partaking in standing overhead press exercises.

Thus, in some embodiments, the mobile computing device 110 may predict the exercise activity type of the user based on a combination of sensor data sets from the smart watch device and based on orientation data sets associated with the mobile computing device 110. For example, when the mobile computing device 110 is in an upstanding position relative to the earth, the user is less likely to be performing bench press exercises when upper body / arm movements are detected. Further, when the mobile computing device 110 is in a position substantially parallel to the earth (e.g., when the user is lying down on a bench with the mobile computing device 110 is in the user’s garment pocket), the user is less likely to be performing standing overhead press exercises. Thus, embodiments of the fitness tracking system described herein may be configured to generate predictions associated with user motion as detected by one or a combination of client devices (e.g., smart watch devices, smart garments, etc.) and to track user motion for generating a series of exercise activity records.

In some embodiments, the mobile computing device 110 may aggregate or combine the series of exercise activity records for storage at a data storage or for transmission to a remote / off-site data server 160. Aggregation of data sets from data-rich computing devices may be the basis for predicting exercise activity based on a plurality of data sets associated with users across user body types, geographies, profiles, or the like. Data sets associated with exercise activities of a pool of users may be used for predicting exercise activities of individual users. Machine learning models of the activity application 212 (FIG. 2) may be iteratively trained and dynamically re-trained for improving exercise activity predictions.

Embodiments of the activity application 212 (FIG. 2) may include operations for detecting type of equipment that a user may be partaking in. As an example, referring again to FIG. 3, the user may be partaking in bench press exercises. In one drawing, the user may be conducting bench presses with a barbell. In another drawing, the user may be conducting bench presses with dumbbells.

It may be beneficial to provide methods of increasing confidence scores / levels of exercise activity predictions based on detection of user motion associated with pre-activity or post-activity. For example, the user may be setting up for conducting bench presses with a barbell, the user may place disc weights at opposing sides of the barbell. The mobile computing device (not explicitly illustrated in FIG. 3) may conduct operations for detecting motion characteristic of a user placing disc weights on opposing sides of the barbell (via sensors on the smart watch device and data sets transmitted to the mobile computing device), such that these detected motion characteristics may be combined with data sets obtained during the actual exercise activity for predicting that the user may be partaking in bench presses with a barbell.

Further, when the user may be handling a barbell for bench press exercises, the mobile computing device may detect that the user motion may suggest the equipment substantially moving along a single axis (e.g., vertically relative to the earth), and may predict that a barbell is being used for exercises.

In contrast, when the user may be setting up for conducting bench presses with dumbbells, the user may pick up respective dumbbells and may exhibit wrist rotation motion to setup the dumbbells in the desired position for the bench press operations. For example, the mobile computing device 110 may conduct operations to identify that equipment being handled based on user motion is about multiple axis, thereby suggesting that dumbbells may be used by the user.

Accordingly, the mobile computing device (not explicitly illustrated in FIG. 3) may conduct operations for detecting motion characteristics of a user rotating dumbbells into a desirable position for bench press exercises, such that these detected motion characteristics may be combined with data sets obtained during the actual exercise activity for predicting that the user may be partaking in bench presses with dumbbells.

In some embodiments, mobile computing devices may be configured to provide at a user interface recommendation for exercise activity based on an associated user’s profile, based on the user’s prior exercise activity logs, or based on externally determined user data. In some embodiments, externally determined user data may include data sets representing user stress levels over time, user sleep quality or sleep patterns, user’s log of recent diet, or user’s log of other physiological data (e.g., any menstrual cycle data, medication usage data, among other examples). Exercise activity recommendations may be based on holistic data associated with the user’s well-being, such as the user’s sleep cycle patterns, records of whether the user is eating healthy meals based on predefined nutrition guidelines. In some embodiments, externally determined data sets may include data associated with historical patterns of the user’s workout routine (e.g., working out leg exercises every Monday, etc.).

In some embodiments, externally determined user data may be obtained based on interfaces with other applications executed on the mobile computing device. For example, the mobile computing device may obtain a user’s menstrual cycle from third-party applications such as Flo, or may obtain a user’s sleep cycle patterns, diet records, heart rate data or blood pressure data from third-party applications or from applications that may be native to the Apple iOS™ environment. In some embodiments, externally determined user data may include the user’s sleep cycle patterns, diet records,

heart rate data or blood pressure data from third-party applications or from applications that may be native to the Android™ environment or other operating system environments.

Based on user data obtained from third party applications, the mobile computing device may be configured to provide recommendations to alter or tweak the user’s daily lifestyle in combination with the user’s exercise activity plans.

In some embodiments, the mobile computing devices may be configured to provide a post-workout analysis for providing workout results, including total volume lifted, average health metrics, among other examples. The post-workout feedback may include recommended future workout routines, followed by recommended diet plans or recovery times.

In some embodiments, the mobile computing devices may be configured to determine whether a user may reach an exercise activity plateau. In some examples, an exercise activity plateau may be identified when the user may not be progressing with the identified exercise activity. In some other examples, an exercise activity plateau may be identified when the user may reach a point of muscle fatigue in their workout, and the user may be no longer able to exercise that muscle group effectively. In some embodiments, machine learning models may be trained to provide recommendations on max weights for repetitions and for best potential weights (e.g., dumbbells) to utilize for maximizing the user’s workout potential.

Referring again to FIG. 1, the fitness tracking platform 100 may include one or more wearable computing devices, such as a smartwatch device 120, an audio device 130, or other wearable computing devices. In some embodiments, the fitness tracking system 100 may be configured to combine data sets from two or more client devices, such as the smartwatch device 120 and the audio device 130, among other wearable devices, to predict an activity type with increased confidence or precision. Such example fitness tracking platforms may be configured to generate exercise activity predictions with increasing confidence or precision.

It may be beneficial to provide a fitness tracking platform configured to generate exercise activity predictions, exercise activity repetition counts, feedback representing exercise form evaluation, among other types of user feedback outputs with increasing confidence or accuracy based on operations of substantially one wearable computing device, such as a smart watch. That is, in some situations, a user may be performing exercises while donning a primary wearable computing device, while leaving other computing devices (e.g., mobile phone, audio headsets, etc.) at other physical locations such that the primary wearable computing device may not be in communication with these other computing devices.

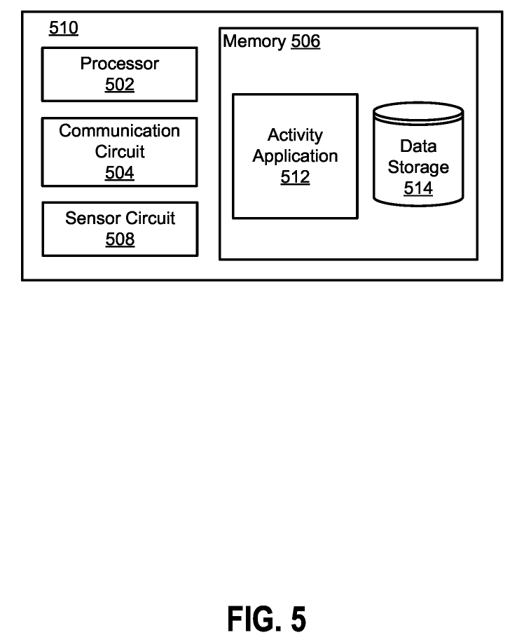

Reference is made to FIG. 5, which illustrates a block diagram of a wearable computing device 510, in accordance with embodiments of the present disclosure. The block diagram of the wearable computing device 510 may be an example smart watch, such as an Apple Watch™, Android™-based smart watch, fitness tracking bands, smart eyewear, smart garments, wireless audio devices, or other type of wearable computing devices. The wearable computing device 510 may be adopted to be worn or donned by a user during one or more exercise activities, such as while working out at a gym or exercising outdoors. The wearable computing device 510 may be configured as a data-rich device, including sensors for detecting motion, patterns inherent in a sequence of motions, identifiable characteristics of detected motion, physical environment conditions, among other sensor-acquired data.

The wearable computing device 510 may include a processor 502, such as a microprocessor or a microcontroller, a digital signal processing processor, an integrated circuit, a field programmable gate array, a reconfigurable processor, or combinations thereof.

The wearable computing device 510 may include a communication circuit 504 configured to transmit or receive data messages to or from other computing devices, to access or connect to network resources, or to perform other computing applications by connecting to a network (or multiple networks) capable of carrying data. The communication circuit 504 may be similar to the communication circuit 204 described with reference to FIG. 2.

The wearable computing device 510 may include memory 506. The memory 506 may store an activity application 512 including processor-readable instructions for conducting one or more operations described herein, such as for conducting machine learning operations associated with exercise type prediction, operations for providing exercise training recommendations in substantial real-time to a user during user exercise activity, operations for evaluating user exercise from, or operations for providing exercise training recommendations in substantial real-time to a user during an exercise activity.

The wearable computing device 510 may include a data storage 614. The data storage 514may be a secure data storage and may store data sets generated by one or more sensor circuits 608.

The one or more sensor circuits 508 may include one or more accelerometers, gyroscopes, pedometers, magnetometers, or barometers, among other examples. The sensor circuit 508 may be configured to generate data sets representing movement or environmental conditions associated with the wearable computing device 510, such as tilt, shake, rotation, acceleration, or swing, among other examples. As will be described, based on one or more identified user movements or physical environment conditions, the wearable computing device 510 may be configured to predict or infer a type of exercise activity being undertaken by a user.

In some embodiments, the wearable computing device 510 may be configured to predict or infer a type of exercise activity in substantial real-time for providing feedback to the user. As an example, a user donning the wearable computing device 510 may conduct pre-exercise activity, such as approaching a dumbbell, lifting the dumbbell, and beginning several repetitions of bicep curls with the dumbbell. Based on sensor data sets generated by the sensor circuit 508, the wearable computing device 510 may be configured to identify the exercise activity prediction (e.g., bicep curl exercise) within several hundred milliseconds, and provide the exercise activity prediction at an output interface within 1 or 2 seconds. Other example time ranges for generating exercise activity predictions and providing the exercise activity prediction at an output interface may be contemplated.

In some embodiments, a series of sensor data generated by a wearable computing device may be configured to generate an exercise prediction based on detected movement of the wearable computing device. For example, when a user wears a smart watch (e.g., Apple Watch™) on their wrist and engages in one or more weightlifting or other conditioning exercises at a fitness gym, the smart watch may be configured to generate a prediction of the exercise type undertaken by the user. For example, the wearable computing device may be configured to generate predictions that a user is conducting bicep curls, bench presses, shoulder presses, among other example exercises.

In some examples, a given exercise may be performed using two or more different types of equipment. For example, bench press exercises may be performed using dumbbells, barbells, or a Smith machine. In another example, bicep curls may be performed using dumbbells or barbells. In another example, shoulder presses may be performed using barbells or a shoulder press machine. It may be desirable to provide fitness tracking devices for generating exercise predictions with greater granularity or precision based on sensor data associated with motion of a user’s limb.

In some embodiments, fitness tracking systems and methods may be configured to provide exercise predictions or exercise activity recommendations to exercise users. Exercise users may have a plurality of varying attributes, including physical size, physiological capacity or capability, health history, or biomarker history, among other attributes. It may be challenging to generate a general baseline exercise activity plan applicable for a spectrum of exercise users. That is, a baseline exercise activity plan may not be a one-size-fits-all exercise activity plan optimal for the respective exercise users. It may be desirable to provide user-specific exercise activity recommendations based on user attributes or biomarker data sets associated with respective users.

In some scenarios, it may be desirable to provide dynamically tuned user-specific exercise activity recommendations during the course of a prior-generated exercise activity planned. As an example, the system may dynamically tune user-specific exercise activity akin to a real-time fitness coach. Based on data sets of sensors, when a user may be slowing down or appear to be unable to finish an exercise set, systems may be configured to provide instruction to reduce intensity or alter exercises. In another example, a user may provide feedback to a system on whether specific equipment is unavailable or that the user’s specific muscles (e.g., biceps) are tired and thereby requesting to focus on a different exercise at the current time. In response, the system may generate an altered user-specific exercise activity recommendation.

In some embodiments, the fitness tracking systems and methods may provide exercise predictions or exercise activity recommendations to exercise users based on large language model-base implementations for providing recommendations based on the current exercise workout and user parameters provided or automatically detected (e.g., repetition time, velocity, etc.) and provide adjusted workout recommendations over time.

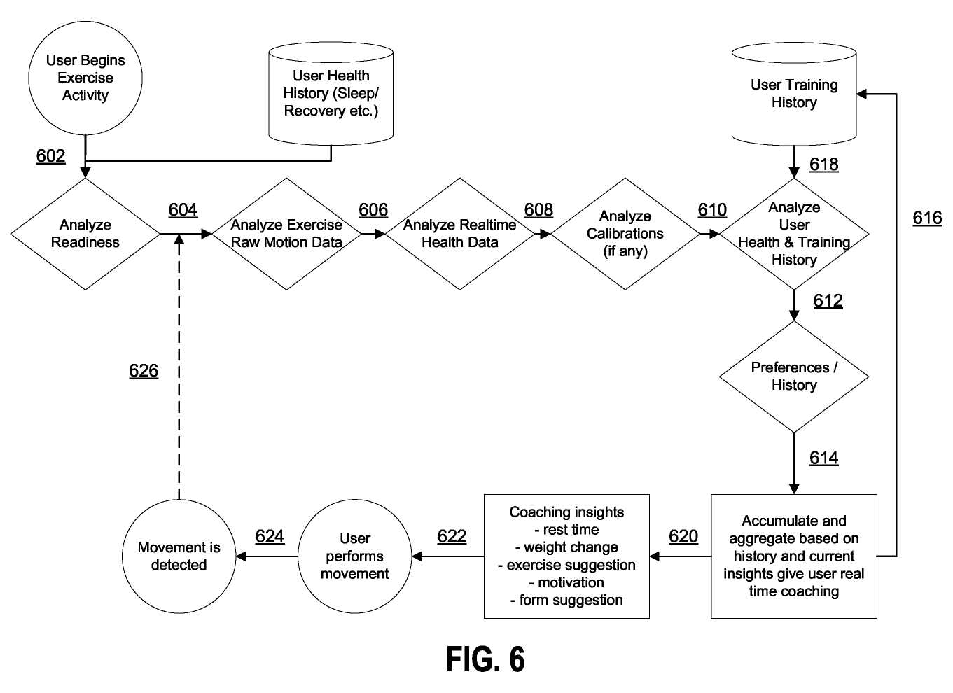

Reference is made to FIG. 6, which illustrates a flowchart of a method 600 of generating exercise activity recommendations, in accordance with embodiments of the present disclosure. In some embodiments, the generated exercise activity recommendations may be configured for generating signals for displaying time-released exercise coaching instructions for the exercise user.

The method 600 may be conducted by the mobile computing device 110 of FIG. 1 (or the mobile computing device 210 of FIG. 1). The processor-executable instructions may be stored in memory and may be associated with the activity application 212 (FIG. 2) or other processor-executable applications not illustrated in FIG. 2. The method 600 may include operations such as data retrievals, data manipulations, data storage, or other operations, and may include computer-executable operations.

The mobile computing device 110 may receive data messages from one or more client devices. For example, the one or more client devices may be wearable computing devices having sensors thereon for detecting motion of the user.

At operation 602, the mobile computing device 110 may receive raw motion data from a client device, such as a wearable computing device. The motion data may be generated based on one or more of gyroscope devices, accelerometer devices, magnetometer devices, among other example devices. The raw motion data may be generated based on a smart watch device donned on the user’s wrist. In some examples, the raw motion data may be generated based on other wearable devices, such as ear-bud devices for audio, among other examples.

In some embodiments, the raw motion data may include time series data representing movement characteristics such as tilting, shaking, rotation, acceleration, stability, velocity, or swinging of the client device over time. The raw motion data may be a proxy representing user movement during exercise activity.

In some embodiments, the raw motion data may include sensor data for representing environmental conditions. For example, the raw motion data may include magnetic field strength or magnetic field direction data associated with equipment that may be nearby the user’s limb. For example, magnetometer sensor data may be for inferring whether a user may be grasping a dumbbell or other equipment. At operation 602, receipt by the mobile computing device 110 of the raw motion data may be associated with initiation of user exercise.

At operation 602, the mobile computing device 110 may retrieve one or more data sets representing user health history, including data sets associated with user sleep or recovery data, pre-existing health conditions, or other user health data associated with the exercise user’s physiological condition.

At operation 604, the mobile computing device 110 may determine exercise activity readiness associated with the exercise user. For example, the mobile computing device 110 may generate prediction values indicating the current physiological condition of the exercise user for conducting a spectrum of user exercises. For instance, the generated prediction values may indicate whether the exercise user may be currently adept for running exercises, may be adept for weight lifting exercises, or may be adept for other types of exercise activity based on the user’s historical attributes representing physiological condition.

In some embodiments, at operation 604, the mobile computing device 110 may generate biometric insights based on physiological user attributes including sleep metric data, stress level data, historical muscle recovery data, menstrual cycle data, or other health-related data associated with the exercise user. As an illustrating example, where the mobile computing device 110 infers that the exercise user has not fully recuperated from an earlier weightlifting session, downstream operations for recommending exercise activity may reduce the intensity or quantity of recommended weightlifting activity.

In some embodiments, the mobile computing device 110 may generate downstream exercise activity recommendations based on various data sets associated with the exercise user’s past exercise activity, the exercise user’s current physiological state, and/or the exercise user’s exercise activity objectives, among other example factors.

At operation 606, the mobile computing device 110 may generate tempo-related insights based on analyzing retrieved raw motion data associated with the exercise user. For example, the generated tempo-related insights may include user velocity data, acceleration data, displacement data, time under tension data, stability data, or tempo data, among other examples.

In some embodiments, the mobile computing device 110 may conduct operations to analyze the retrieved raw motion data based on a machine learning model or advanced signal processing. In some embodiments, machine learning models may include operations to identify repetition characteristics such as where repetitions start, end, or pause. In some embodiments, advanced signal processing may include operations to include sensor fusion operations for estimating velocity displacement or other insights and may include additional models for fine-tuning velocity displacement estimates or the like.

At operation 608, the mobile computing device 110 may generate health-related insights based on health-data associated with the exercise user. For example, the generated health-related insights may include peak heart rate data, heart rate variability data, respiratory rate data, or strain data, among other examples.

At operation 610, the mobile computing device 110 may generate form analysis insights based on calibration data associated with the exercise user. For example, the generated form analysis insights may include data representing a quality metric of the user exercise or data representing whether there may be muscular imbalance.

In some embodiments, the mobile computing device 110 may be configured to generate feedback prompts to the exercise user for correcting exercise form. For instance, exercise form may be related to alignment of user limbs during exercise activity. In some embodiments, feedback prompts may be audible feedback prompts, haptic feedback prompts, or visual feedback prompts.

At operation 612, the mobile computing device 110 may generate data sets representing meta metrics associated with the user conducting the exercise activity. For example, the mobile computing device 110 may generate meta metrics representing values for indicating relative effort undertaken by the exercise user, exertion by the exercise user, or repetition maximum percentage associated with the user, among other examples. Generating the meta metrics may be based on data sets associated with the user health history or exercise user training history. The generated meta metrics may be desirable for monitoring an exercise user’s resistance training intensity, as an example.

At operation 614, the mobile computing device 110 may determine user-focused exercise activity preferences based on data sets representing the user’s past exercise goals, the user’s past injuries list, or the user’s past personal exercise preferences.

At operation 616, the mobile computing device 110 may generate exercise recommendation plans for providing exercise coaching instructions based on a combined analysis of user attributes and the raw motion data. In some embodiments, such exercise recommendation plans may be provided as coaching prompts in substantial real-time and provided over a duration of time.

At operation 618, the mobile computing device 110 may store the generated exercise recommendation plan in a user training history for downstream operations directed to analyzing user health and user training history.

At operation 620, the mobile computing device 110 may transform or combine the generated exercise recommendation plans with insights based on prior identified tempo-related insights, health-related insights, form analysis, or meta metrics, among other types of data sets.

At operation 622, the mobile computing device 110 may generate exercise coaching insights. In some embodiments, exercise coaching insights may include data sets representing rest time of the exercise user, weight change of the exercise user, exercise suggestions received from the exercise user, motivation indicators associated with the exercise user, or physical form suggestions for the exercise user.

At operation 624, the mobile computing device 110 may continually receive raw motion data from the wearable computing device whilst the exercise user may be conducting one or more exercise activities.

At operation 626, the mobile computing device 110 may be configured to iterate on the analysis, predictions, or generation of exercise activity recommendations for the exercise activity user.

Embodiments of the present disclosure include systems and methods of generating user-focused exercise activity recommendations and plans based on a cumulative and combinatory analysis of raw motion data representing user movements, user health history data, user biometric insights obtained from prior points in time, and/or iteratively generated user activity insights over time.

As an example, user-focused exercise activity recommendations may include recommended exercises, recommended rest times, set counts, repetition counts, weight quantity, weightlifting style (e.g., bench press activity versus barbell activity), among other examples of exercise activity plans.

In some embodiments, a fitness tracking system can generate multi-week coaching plans that build out daily workout routines tailored to the user’s specific needs and performance data. By analyzing motion data, such as range of motion captured through a smartwatch, the system can identify deficiencies in exercise execution and incorporate corrective measures into the plan. For example, if the system detects that a user’s deadlift lacks full range of motion, potentially due to tight hamstrings, it can recommend adding targeted hamstring stretches to improve flexibility. Similarly, if acceleration data shows that the user’s incline bench press is slower compared to their regular bench press, the system may infer that the upper pectoral muscles are relatively weaker than the lower pectorals and adjust the plan to include exercises focused on strengthening the upper chest.

The artificial intelligence model(s) implemented by described embodiments of fitness tracking systems herein can include any suitable type of AI model or combination of models capable of processing motion data, generating exercise-related insights, and providing exercise recommendations based on said insights. Examples of AI models that may be used include one or a combination of large language models (LLMs), convolutional neural networks (CNNs), recurrent neural networks (RNNs), long short-term memory networks (LSTMs), transformers, decision trees, support vector machines (SVMs), and ensemble models. The model may be trained to recognize patterns in motion data and correlate those patterns with specific exercises, repetition counts, movement quality indicators, and effort metrics. The AI model(s) can be executed entirely on a single computing device, such as a server or a mobile computing device (e.g., a smart phone), or its computational workload can be distributed across multiple devices. For example, portions of the model may be run on a server, a mobile computing device, and one or more wearable computing devices (e.g., a smart watch), enabling efficient real-time processing and responsiveness while leveraging the capabilities of each device in the system.

Some fitness tracking system embodiments can use a combination of AI models, each configured to perform a different set of tasks. In some implementations, the combination of AI models can include a first AI model configured to detect and assess exercise movements in real time using motion data captured by wearable devices associated with the user, such as smartwatches, fitness bands, or wireless headphones. This evaluation can include an assessment of movement quality, such as an assessment of the user’s form while performing an exercise, as well as an assessment of user effort, such as a gauge of how strenuous the exercise was for the user. Output from the exercise activity assessment model can then be provided to a second AI model configured to process the output to generate tailored exercise recommendations for the user.

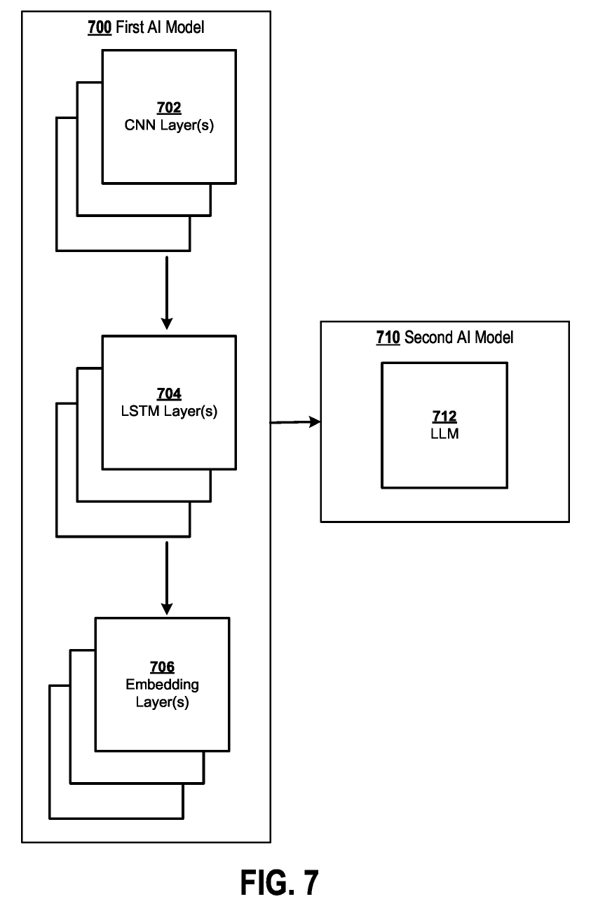

A diagram of an example implementation of an AI model architecture that can be implemented by the described fitness tracking systems is provided in FIG. 7. As shown, the AI model architecture can include a first AI model 700 and a second AI model 710.

In some embodiments, the first AI model 700 can be implemented on a first device or computer system and the second AI model 710 can be implemented on a second device or computer system. For example, the first AI model 700 can be implemented on a smart phone and the second AI model 710 can be implemented on a remote server. In other embodiments, the first AI model 700 and the second AI model 710 can be implemented on the same device or computer system.

The first AI model 700 can include one or more convolutional neural network (CNN) layers 702, one or more long short-term memory (LSTM) layers 704, and one or more embedding layers 706. The second AI model 710 can include at least one LLM layer 712. Each of these layers is described in further detail below.

The CNN layer(s) 702 can include any suitable number or combination of CNNs. Each CNN can be a deep learning model configured to scan data provided as input to the CNN with convolutional filters that detect local patterns in the data. The CNN layer(s) 702 can allow the first AI model 700 to learn and generalize complex patterns in raw motion data provided by sensors in a wearable computing device (e.g., a smart watch) and thereby enable the AI model 700 to identify spatial and temporal correlations in the raw motion data.

In some embodiments, the CNN layer(s) 702 can be configured to receive data characterizing raw motion of a user (e.g., accelerometer or gyroscope data provided by one or more sensors of a wearable computing device) as input. The CNN layer(s) 702 can transform this raw motion data to identify motion features, assign weights to the identified features based on learned parameters, and store those features for further processing by downstream components of the model. This architecture can enable efficient and scalable analysis of motion data and can support accurate and real-time recognition of exercise movements.

In some embodiments, the LSTM layer(s) 704 can be downstream of the CNN layer(s) 702 and can include any suitable number or combination of LSTM networks. Each LSTM network can be a recurrent neural network (RNN) configured to learn and retain information across time steps. Each LSTM layer 704 can incorporate memory cells and gating mechanisms that enable the AI model 700 to selectively retain or discard information, allowing it to capture long-range dependencies and temporal patterns in motion data. In some embodiments, the LSTM layer(s) 704 can be configured to receive motion features extracted by preceding layers (e.g., the CNN layer(s) 702) and analyze how those features evolve over time. This temporal analysis can support accurate identification of exercise repetitions, assessment of movement consistency, and evaluation of effort dynamics throughout a workout session.

In some embodiments, the embedding layer(s) 706 can be downstream of the LSTM layer(s) 704 and can include any suitable number or combination of embedding models. Each embedding layer can be configured to transform categorical or high-dimensional input data into dense, lower-dimensional vector representations. The embedding layer(s) 706 can enable the AI model 700 to efficiently encode contextual information, such as user-specific physiological attributes (e.g., height, weight, gender), exercise types, or device-specific sensor characteristics. These embeddings can allow the model 700 to incorporate personalized context into its analysis, improving its ability to generalize across users while maintaining sensitivity to individual differences.

In some embodiments, the first AI model 700 can generate quantitative metrics that characterize both the quality of a user’s movements and the level of effort exerted during exercise. In some embodiments, for each set of repetitions performed by the user, the model derives an estimated effort score based on raw motion data (e.g., motion data captured by wearable devices). To derive the estimated effort score, the model 700 can analyze parameters such as acceleration and velocity of the user (or a body part of the user) for each repetition. strong acceleration and velocity may indicate good performance, while declines in acceleration and velocity may signal fatigue and increased exertion. Based on this analysis, the model 700 can assign an effort score to the set that reflects that the amount of effort that the user had to exert to complete the repetitions.

In addition to assessing sets of repetitions of an exercise performed during a workout session, the first AI model 700 can be configured to evaluate a workout session as a whole. In some embodiments, to determine how strenuous an entire workout was, the AI model 700 can aggregate effort scores across all sets performed during the session. From these aggregated values, the AI model 700 can derive a strain score for the workout, which can be further applied to estimate muscle fatigue for individual muscle groups. This can enable the fitness tracking system to provide a holistic view of workout intensity and its physiological impact on the user.

In addition to evaluating user effort, the first AI model 700 can, in some implementations, determine one or more motion-based metrics for an exercise activity or workout session, including absolute values and/or changes over time for acceleration, velocity, range of motion or displacement, time under tension, power, and motion path stability. The model can also evaluate and incorporate physiological signals such as heart rate, heart rate variability, and body temperature. These parameters can enable the fitness tracking system to provide a comprehensive assessment of exercise performance and user exertion and form a robust basis for generating personalized recommendations.

As shown, the second AI model 710 can be downstream of the first AI model 700. The second AI model 710 can receive data regarding an exercise activity, a workout session, or a combination thereof from the first AI model 700. This data can include, for example, one or more effort scores associated with one or more exercise activities, one or more strain scores associated with one or more workout sessions, one or more motion-based metrics, or combinations thereof. The second AI model 710 can use the data provided by the first AI model 700 to provide customized exercise recommendations that are tailored to facilitate user progress toward personal fitness goals.

The at least one LLM layer 712 of the second AI model can include any suitable number of LLM layers. Each LLM layer can be built upon a neural network architecture such as a deep neural network or a recurrent neural network (RNN) architecture, for example, a transformer deep neural network architecture. The LLM layer(s) 712 can enable the second AI model to efficiently provide exercise recommendations in a human-readable (e.g., natural language) format.

In some embodiments, exercise recommendations can be delivered in the form of interactive templates within an application associated with the fitness tracking system. Each template can represent a structured workout plan that includes details such as exercises to be performed, prescribed rest periods, number of sets, and target repetitions. Users can initiate a workout by selecting a template and then selecting a “Start” option within the application, which launches the guided workout session. This format can provide a convenient and user-friendly way for individuals to follow personalized exercise plans generated by the system.

In some embodiments, the LLM layer 712 can be configured to output exercise recommendations in a machine-readable format or language. For example, the LLM layer 712 can produces a JSON output that conforms to a predefined schema required for integration with the associated application. In some embodiments, a workout template corresponding to the exercise recommendation that is displayed to the user in an application associated with the fitness tracking system can be provided based on the machine-readable LLM output.

In some embodiments, a library of predefined prompts for interacting with the LLM layer 712 can be employed. The prompts can be based on specific user actions within an application associated with the fitness tracking system. For example, when a user taps a button to generate a workout, a corresponding prompt from the library can be retrieved and passed to the LLM layer 712 to produce an appropriate recommendation. Additionally, or alternatively, user input can be provided by the user directly to the LLM layer 712 via a chat interface. This direct LLM interfacing functionality can enable users to, for example, request customized guidance or adjustments to their workout plan.

The AI model architecture embodiment illustrated in FIG. 7 is presented for illustrative purposes and is not intended to be limiting. An AI model implemented by a fitness tracking system can include a different combination of model types than the combination illustrated in FIG. 7, and can be configured with any number of layers, nodes, weights, biases, activation functions, and other parameters. The architecture of an AI model can be selected or adapted based on the nature of the motion data being processed, the desired output metrics, and the computational resources available, allowing for flexible and scalable implementation across a variety of fitness tracking scenarios.

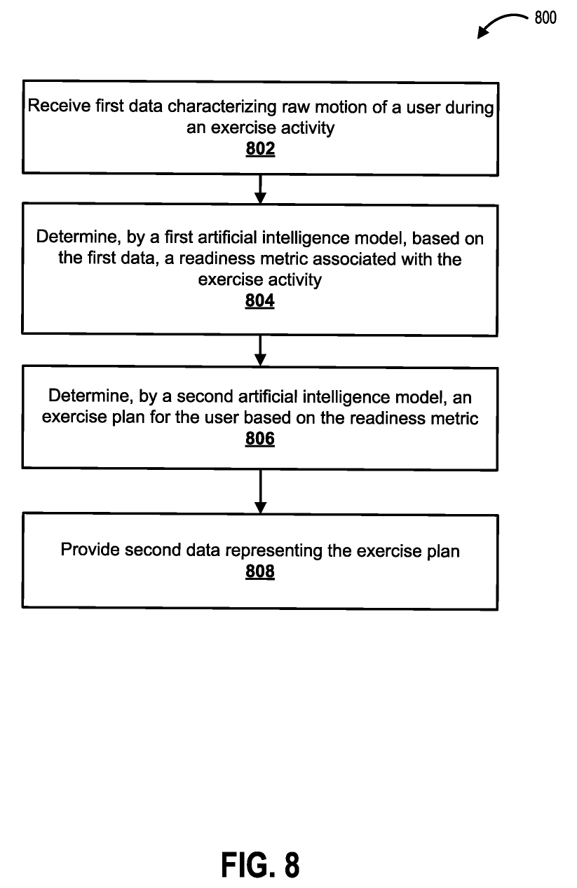

Reference is now made to FIG. 8, which illustrates a flowchart of a method 800 of generating fitness exercise recommendations based on machine learning architecture, in accordance with embodiments of the present disclosure. The method 800 can be performed, all or in part, by one or more processors of components of a fitness tracking system, for example, processor(s) of a mobile computing device (e.g., a smart phone), a wearable computing device (e.g., a smart watch), and/or other computing devices (e.g., a server) that constitute a fitness tracking system. In some embodiments, the method 800 can be implemented as instructions stored in non-transitory memory. Alternatively, or in addition, the method 800 can be included in non- transitory computer readable memory storing the method 800 as instructions which, when executed by one or more processors forming part of a fitness tracking system, causes the processor(s) to perform operations of the method 800.

In some embodiments, the method 800 may include operations conducted by a processor of one or a combination of a wearable computing device worn on a user limb or by the mobile computing device 110 (FIG. 1). For example, a wearable computing device may be a smart watch device or a fitness tracking band configured to be donned on a user’s wrist. The wearable computing device may be the wearable computing device 510 of FIG. 5.

In some embodiments, the method 800 may include operations conducted by one or more processors of the wearable computing device or the mobile computing device and may include operations such as data retrievals, data manipulations, data storage, or other operations, and may include computer-executable operations.

In some embodiments, the wearable computing device configured to be worn on a user limb, such as a user’s wrist, may include a sensor circuit configured to generate sensor data. The sensor circuit may include one or more of accelerometers, gyroscopes, pedometers, magnetometers, or barometers, among examples of sensor devices.

As described, the wearable computing device may include one or more sensors for detecting movement or other environmental conditions and may generate a sequence or series of sensor data over time (e.g., time-series sensor data set). The fitness tracking device may store the sensor data for generating exercise recommendation routines and for iteratively updating the exercise routine based on iteratively received data associated with the exercise user.

As described in the present disclosure, it may be challenging to generate a baseline exercise plan for exercise activity (e.g., weightlifting, cardio-based activity, etc.) for a spectrum of exercise users at least because exercise users may have a plurality of varying attributes, including physical size, physiological capacity or capability, health history, or biomarker history, among other attributes.

Some embodiments of the present disclosure may include systems and methods to provide user-specific exercise activity recommendations that may be iterated over time, or in substantial real-time during exercise activity) based on user attributes or biomarker data sets associated with the respective users.

At operation 802, the processor may receive first data characterizing raw motion of a user during an exercise activity. In some embodiments, the first data may include time series data representing movement characteristics of the exercise user donning a wearable computing device. In some embodiments, the processor may determine a predicted exercise activity based on the raw motion data and an indicated start of such predicted exercise activity.

In some implementations, at 802, the first data characterizing raw motion of the user is received from one or more sensors of one or more wearable computing devices of a user. The wearable computing device(s) can include a smart watch, a fitness tracking band, wireless headphones or earphones, and/or the like. The sensor(s) of the wearable computing device(s) can include accelerometers, gyroscopes, magnetometers, or other motion- sensing components capable of capturing physical movement. The first data can be received by another computing device, for example, a mobile computing device (e.g., a smart phone), a personal computer, or a server that is communicatively coupled to the wearable computing device(s). In some implementations, the first data can be transmitted from the wearable computing device(s), for example, over a wireless communication network (e.g., a Bluetooth connection), with minimal latency (e.g., less than one second from measurement), enabling timely and responsive analysis of user activity.

In some implementations, the first data includes a time series of vectors representing how the user (or a specific body part of the user, such as the user’s wrist or head) is accelerating in three-dimensional space. Each vector in the time series can encode both the magnitude and direction of acceleration at a given time step, providing a detailed representation of the user’s motion over time. The size of the first data set may vary depending on the sampling frequency of the sensors, which may range from tens to hundreds of measurements per second.

In some embodiments, the first data can include data characterize user motion during an exercise activity as well as during periods of time preceding and following the exercise activity. For example, the first data can include motion data for approximately five seconds (or another duration) before and five seconds (or another duration) after an exercise is performed to provide additional context for exercise detection. This pre- and post-exercise motion data can indicate movements such as placing weights on a barbell, picking up dumbbells, walking to or from a squat rack, sitting down or lying on a bench, or other preparatory or concluding actions. Although these movements are not part of the exercise itself, they can provide valuable clues that can improve the accuracy of exercise identification by indicating the type of equipment being used and the user’s intended activity.

Upon receipt at 802, the first data can be provided as input to a first AI model implemented by the fitness tracking system that is configured to detect, identify, and assess exercise activities. An example implementation of such an AI model is the first AI model 700 described with respect to FIG. 7. In some embodiments, the first data can be provided to the first AI model without undergoing any preprocessing. This can enable the model to learn and extract relevant features from raw motion signals and thereby preserve the full fidelity and variability of the data, which can improve generalization and adaptability across diverse users and movement styles. In other embodiments, the first data can be preprocessed (e.g., filtered, smoothed, normalized, etc.) prior to being provided as input to the first AI model. Preprocessing the first data can reduce noise, enhance signal clarity, and improve model stability and performance by presenting cleaner, more consistent inputs for feature extraction and prediction.

To guide the exercise user with the identified predicted exercise activity, the processor may conduct operations for generating a user-focused recommended exercise plan for the exercise user.

At operation 804, a readiness metric associated with the exercise activity is determined, based on the first data, by the first AI model. The readiness metric can include data that characterizes an ability of the user to perform different exercise activities.

In some embodiments, determining the readiness metric can involve determining an effort score for the exercise activity that reflects how strenuous the exercise activity was for the user. In some embodiments, determining the readiness metric can involve determining a strain score that indicates an amount of effort expended by the user during a workout session in which the exercise activity was performed. In some embodiments, determining the readiness metric can involve determining one or more motion metrics associated with the exercise activity, such as metrics characterizing the acceleration, velocity, range of motion or displacement, time under tension, power, and/or motion path stability of the user (or a body part of the user) during the exercise activity. In some embodiments, the readiness metric can be a data structure (e.g., an array) that contains one or more of the effort score, the strain score, and the motion metrics. In other embodiments, the readiness metric can be a value or a data structure that is derived based on one or more of the effort score, the strain score, and the motion metrics.

In some implementations, determining the readiness metric can involve determining one or more physiological characteristics of the user. In some embodiments, these characteristics can be identified using the first AI model. For example, the first AI model may utilize information learned during previous fitness tracking sessions to infer physiological characteristics based on historical motion data and user behavior. Additionally, or alternatively, data regarding physiological characteristics of the user can be stored by one or more computing devices of the fitness tracking system, such as the wearable computing device or a mobile computing device, and accessed by the first AI model during analysis. In some embodiments, data regarding the user’s physiological characteristics may be provided directly by the user through manual input.

The determined physiological characteristics can be any characteristics that influence how the user moves while exercising. Examples of such physiological characteristics include (but are not limited to) the user's height, weight, age, gender, limb length, body composition, and overall fitness level, all of which can affect the range of motion, acceleration profiles, and movement dynamics of the user.

In addition to determining static physiological characteristics, in some implementations, a current physiological state of the user can be assessed. Assessing the current physiological state of the user can involve determining (e.g., by the first AI model and/or by accessing data stored by one or more computing devices of the fitness tracking system) information such as the amount of sleep or rest the user has had over a recent period (e.g., the preceding 24 hours), whether the user is menstruating, the user's hydration level, the user’s nutritional intake (e.g., the amount of protein, carbohydrates, fats, or other nutrients consumed recently), and/or the like. Other examples of physiological state data may include stress levels, recent illness or injury, medication usage, and environmental factors such as ambient temperature or altitude. These dynamic factors can influence the user's movement patterns and effort output and can be considered by the AI model to improve the accuracy and personalization of exercise recognition and performance evaluation.

In some embodiments, the readiness metric may be generated based on prior-conducted exercise activity data associated with the user. In some scenarios, biometric insights associated with the exercise user may have an impact on whether the exercise user may competently or safely conduct the predicted exercise activity. To illustrate, in a scenario where the exercise user may have had an intensive weightlifting session in a prior day, the biometric insights may determine that the exercise user’s muscles have not adequately recovered for a subsequent intensive weightlifting session. Accordingly, the processor may generate a readiness metric associated with a subsequent intensive weightlifting session to indicate that the exercise user may not be able to conduct a heavy series of weight exercises.

Upon its determination at 804, the readiness metric can be provided as output by the first AI model and subsequently input to a second AI model implemented by the fitness tracking system that is configured to provide the user with exercise activity recommendations. An example implementation of such an AI model is the second AI model 710 described with respect to FIG. 7. In some embodiments, the readiness metric can be provided to the first AI model without undergoing any preprocessing or other transformations. In other embodiments, the readiness data can be preprocessed or otherwise transformed (e.g., filtered, smoothed, normalized, etc.) prior to being provided as input to the second AI model.

At operation 806, an exercise plan for the user is determined, by the second AI model, based on the readiness metric. Determining the exercise plan can involve prompting an LLM of the second AI model (e.g., the LLM 712 described with respect to FIG. 7) to generate an exercise plan for the user in view of the readiness metric.

In some embodiments, the exercise plan can be a plan for a current workout session. An exercise plan for a current workout session can include (for example) suggestions for additional exercise activities to be performed during the workout session, suggestions regarding a number of repetitions that the user should execute in a given set of an exercise activity, suggestions regarding an amount of weight or resistance that the user should use during an exercise activity, and/or the like can be provided. The fitness tracking system can be configured to continuously update and adjust such exercise plans over the course of the workout session to optimize user progress during the workout session.

In some embodiments, the exercise plan can be a plan for a future workout session. An exercise plan for a future workout session can include indications of muscle groups to target during the future workout session, one or more exercise activities to be performed during the future workout session, suggestions regarding an amount of weight or resistance that the user should use during each recommended exercise activity, and/or the like.

Some implementations of fitness tracking systems may, at 806, generate an activity data set representing a recommended exercise plan. Continuing with the weightlifting exercise example, in a scenario where the exercise user may appear to wish to conduct a weightlifting session (today) following a prior intensive weightlifting session, the fitness tracking system (or a processor thereof) may generate an activity data set that may modulate or temper the intensity of a subsequent weightlifting session. The generated activity data set may be a recommended exercise plan that reduces the intensity of the subsequent weightlifting session based on the exercise user’s current physiological state. The exercise user’s current physiological state may be based on the user’s prior sleep quality, stress levels, muscle recovery assessments, or other physiological status.

In some embodiments, the fitness tracking system (or a processor thereof) may iteratively alter the recommended exercise plan over time based on substantial real-time determination of the exercise user’s tempo-related insights (e.g., exercise velocity, displacement, time under tension, etc.), the exercise user’s run-time vitals (e.g., heart rate, respiratory rate, strain data, etc.), the exercise user’s predicted form (e.g., alignment of user limbs as compared to ideal exercise metrics, etc.), or historical data representing the exercise user’s prior repetitions of such exercises.

In some embodiments, the fitness tracking system (or a processor thereof) may alter the recommended exercise plan over the course of a particular exercise set by the exercise user based on run-time user vitals or other user-focused insights. For example, if the processor identifies that the exercise user’s heart rate is higher than expected or higher than a healthy level, the processor may alter the recommended exercise plan to guide the exercise user to decrease intensity of the particular exercise activity. As an example, the higher-than-expected user heart rate may be associated with inadequate user sleep or other physiological state at the present time.

In some embodiments, the recommended exercise plan may include at least one or more parameters associated with exercise activity type, time between exercise sets, exercise set count, repetition count, weight quantity, or exercise style. In some embodiments, the recommended exercise plan may include time / distance for exercises such as a “Farmer’s walk”, in which a weighted implement is deadlifted and carried for a particular recommended distance.

At operation 808, second data representing the exercise plan can be provided. Providing the second data can involve transmitting the data over a wireless communication network (e.g., Bluetooth, Wi-Fi, or cellular) to one or more computing devices of the fitness tracking system, such as a wearable computing device (e.g., a smart watch) or a mobile computing device (e.g., a smart phone). In some embodiments, the second data (or a visual representation thereof) can be displayed on a graphical user interface (GUI) of an application associated with the fitness tracking system. The GUI can present the exercise plan on the display of the wearable device, the mobile device, and/or another computing device connected to the system.

In some embodiments, the second data can be provided together with a request for user input regarding the exercise plan. The request can prompt the user to, for example, accept the exercise plan, reject the exercise plan, or adjust aspects of the exercise plan. The AI model can be configured to use such user feedback to improve future exercise recommendations through retraining or adjustment of model parameters, thereby enhancing accuracy and personalization over time.

In some embodiments, the second data can include a signal for displaying the recommended exercise plan on a display over time. For example, the second data can include a signal for displaying guidance to the user through the duration the user conducting the exercise activity. The displayed guidance may include guidance associated with time between exercise sets, exercise set counts, exercise repetition counts, weight quantity where the exercise activity is associated with weights or other equipment, or exercise style.

The term “connected” or "coupled to" may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references may be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

What is claimed is:

1. A system comprising:
at least one processor; and
non-transitory memory storing instructions which, when executed by the at least one processor, cause the at least one processor to perform operations comprising:
receiving, from one or more wearable devices comprising one or more sensors for detecting motion of a user, first data characterizing raw motion of a user during an exercise activity;
determining, using a first artificial intelligence (AI) model, based on the first data, a readiness metric associated with the exercise activity;
retrieving an exercise plan for the user based on the readiness metric generated based on a second AI model; and
providing second data characterizing the exercise plan.

2. The system of claim 1, wherein the one or more wearable devices comprise a smart watch, a fitness tracking band, wireless headphones, or wireless earphones.

3. The system of claim 1, wherein the at least one processor comprises a first processor associated with a first device and a second processor associated with a second device, wherein:
the AI model is implemented by the first processor; and
the second AI model is implemented by the second processor.

4. The system of claim 3, wherein the first processor and the second processor are communicatively coupled over a wireless network.

5. The system of claim 1, wherein the first AI model comprises one or more convolutional neural network (CNN) layers, one or more long short-term memory (LSTM) layers, one or more embedding layers, or a combination thereof.

6. The system of claim 1, wherein the second AI model comprises at least one large language model (LLM) layer.

7. The system of claim 1, wherein the operations further comprise:
determining, by the first AI model, one or more physiological characteristics of the user;
wherein the readiness metric is further determined based on the one or more physiological characteristics.

8. The system of claim 1, wherein determining the readiness metric comprises:
determining an effort score representing an amount of effort exerted by the user during the exercise activity.

9. The system of claim 1, wherein determining the readiness metric comprises:
determining one or more motion metrics characterizing motion of the user or a body part of the user during the exercise activity.

10. The system of claim 9, wherein the one or more motion metrics comprise metrics indicating an acceleration of the user or the body part, a velocity of the user or the body part, a time under tension of the user or the body part, a power exerted by the user or the body part, a motion path traversed by the user or the body part, a stability of the user or the body part, or a combination thereof.

11. A computer-implemented method comprising:
receiving, from one or more wearable devices comprising one or more sensors for detecting motion of a user, first data characterizing raw motion of a user during an exercise activity;
determining, using a first artificial intelligence (AI) model, based on the first data, a readiness metric associated with the exercise activity;
retrieving an exercise plan for the user based on the readiness metric generated based on a second AI model; and
providing second data characterizing the exercise plan.

12. The method of claim 11, wherein the one or more wearable devices comprise a smart watch, a fitness tracking band, wireless headphones, or wireless earphones.

13. The method of claim 11, wherein the first AI model is implemented by a first processor associated with a first device and the second AI model is implemented by a second processor associated with a second device.

14. The method of claim 11, wherein the first AI model comprises one or more convolutional neural network (CNN) layers, one or more long short-term memory (LSTM) layers, one or more embedding layers, or a combination thereof.

15. The method of claim 11, wherein the second AI model comprises at least one large language model (LLM) layer.

16. The method of claim 11, further comprising:
determining, by the first AI model, one or more physiological characteristics of the user;
wherein the readiness metric is further determined based on the one or more physiological characteristics.

17. The method of claim 11, wherein determining the readiness metric comprises:
determining an effort score representing an amount of effort exerted by the user during the exercise activity.

18. The method of claim 11, wherein determining the readiness metric comprises:
determining one or more motion metrics characterizing motion of the user or a body part of the user during the exercise activity.

19. The method of claim 18, wherein the one or more motion metrics comprise metrics indicating an acceleration of the user or the body part, a velocity of the user or the body part, a time under tension of the user or the body part, a power exerted by the user or the body part, a motion path traversed by the user or the body part, a stability of the user or the body part, or a combination thereof.

20. A non-transitory computer readable storage medium storing instructions which, when executed by at least one processor, cause the at least one processor to perform operations comprising:
receiving, from one or more wearable devices comprising one or more sensors for detecting motion of a user, first data characterizing raw motion of a user during an exercise activity;
determining, using a first artificial intelligence (AI) model, based on the first data, a readiness metric associated with the exercise activity;
retrieving an exercise plan for the user based on the readiness metric generated based on a second AI model; and
providing second data characterizing the exercise plan.
